(19) United States
(12) Patent Application Publication
(10) Pub. No.: US 2024/0321459 A1
(43) Pub. Date: Sep. 26, 2024

(54) SYSTEM AND METHOD FOR ASSESSING RISK PREDISPOSITION TO GESTATIONAL HYPERTENSIVE DISORDERS AND DEVELOPING PERSONALIZED NUTRITION PLANS FOR USE DURING STAGES OF PRECONCEPTION, PREGNANCY, AND LACTATION/POSTPARTUM

(71) Applicant: Ali Mostashari, New York, NY (US)

(72) Inventors: Ali Mostashari, New York, NY (US); Raya Khanin, New York, NY (US); Klemo Vladimir, New York, NY (US); Mario Storga, New York, NY (US); Marija Majda Perisic, New York, NY (US)

(73) Assignee: LifeNome Inc., New York, NY (US)

(21) Appl. No.: 18/210,127

(22) Filed: Jun. 15, 2023

Related U.S. Application Data
(60) Provisional application No. 63/453,332, filed on Mar. 20, 2023.

(51) Int. Cl. G16H 50/30; G16H 10/20; G16H 20/60; G16H 50/20

(52) U.S. Cl. CPC G16H 50/30; G16H 10/20; G16H 20/60; G16H 50/20

(57) ABSTRACT

A system for computing risk predisposition to gestational hypertensive disorders for an individual woman is provided. The system comprises a computer and an application stored in the computer that when executed computes, based on received data, risk predisposition to gestational hypertensive disorders for a female. The system also calculates, based at least on the computed risk predisposition, calories, and macro-and micro-nutrient needs for the female. The system also generates, based on the calculations, dietary recommendations, foods, and recipes for the female. The system further receives feedback from a plurality of females regarding liking/disliking of at least previous dietary recommendations comprising foods and recipes and receives further feedback regarding adverse reactions comprising at least one of morning sickness and nausea. The system further receives additional data comprising at least one of pregnancy complications, blood pressure, and glucose levels.

### CROSS REFERENCE TO RELATED APPLICATIONS

None

### FIELD OF THE DISCLOSURE

The present disclosure is in the field of assessing predisposition to gestational hypertensive disorders during preconception or early pregnancy with an objective of reducing risk and improving maternal and child health through lifestyle and nutritional modifications. More particularly, the present disclosure provides systems and methods that use combinations of women's genomics data (DNA, RNA), self-reported data, and data from wearables to identify women at early stages of elevated risk of gestational hypertensive disorders and personalize dietary recommendations and meal plans to reduce risks of such disorders.

### BACKGROUND

Gestational hypertensive disorders (GHDs), also known as hypertensive disorders of pregnancy, are prevalent complications of pregnancy. The GHDs include preeclampsia, defined as new-onset hypertension or worsening hypertension after 20 weeks gestation plus proteinuria or other evidence of end-organ dysfunction; gestational hypertension, defined as new-onset hypertension without accompanying features of preeclampsia and eclampsia. Eclampsia is defined as the progression of preeclampsia to maternal seizures. Research indicates that GHDs occur in 5.2-8.2% of all pregnancies (Ref. 1). GHDs are a leading cause of maternal and perinatal morbidity and mortality worldwide. Approximately one-quarter of global maternal deaths result from pre-eclampsia (Ref. 2).

Incidences of preeclampsia in the US rose by 25% between 1987 and 2004. Since 1980, the risk of severe preeclampsia has increased by more than sixfold (Ref. 3). A decade ago, the burden of pre-eclampsia including treatments within the first twelve months of delivery was estimated to be $2.18 billion. Premature births bore a disproportionate share of the cost of $1.03 billion for women and $1.15 billion for infants. (Ref. 4).

Preeclampsia is a serious pregnancy complication that is characterized by new-onset hypertension typically after twenty weeks of gestation and is frequently observed near term. Intrauterine growth restriction is a common manifestation of this condition, often necessitating early delivery of the infant. If left untreated, maternal hypertension may cause seizures, brain bleeding (hemorrhagic stroke), coma, or hypertensive encephalopathy. Fetal impairment of blood and oxygen flow can also result from severe preeclampsia, leading to growth complications or even stillbirth.

The standard screening test for GHD has traditionally been measuring blood pressure, which is often only effective when the disease has already begun to progress. In such cases, it becomes crucial to consider risk factors to evaluate the likelihood of developing GHD. Women with pre-existing health conditions such as high BMI, adiposity, hypertension, heart disease, diabetes, or kidney disease before pregnancy have a higher risk of developing GHD, and preeclampsia.

Other moderate risk factors for developing GHD include advanced maternal age and a family history of pre-eclampsia. Although these criteria may not be difficult to apply, they perform with poor sensitivity (Ref. 5). Prediction performance for late gestation preeclampsia is even less accurate, further demonstrating the inefficient nature of these diagnostic criteria.

More accurate clinical tests are available to predict preterm GHD or preeclampsia, but due to the high costs of implementation, universal access is difficult to implement. (Ref. 6). A significant unmet need remains for predictive early screening tests to reduce the disease burden associated with GHD.

Research suggests that genetics contributes to the risk of developing preeclampsia and gestational hypertensive pregnancy complications. Large-scale genetic studies estimate that heritability for GHD, including preeclampsia, is approximately 55% (Ref.7). Specific genetic variants encoding components of the renin-angiotensin system, coagulation, fibrinolysis, lipid metabolism, and inflammation increase the risk of developing GHDs, including preeclampsia. Many genetic variations that contribute to the risk of GHDs are also linked to an increased risk of cardiovascular diseases (Ref. 8).

Earlier studies have explored a limited number of genes involved in the molecular mechanisms of GHDs. It is essential to use large-scale genomics data to identify genetic variations associated with the risk of GHDs, which are complex, and likely heterogeneous diseases. To this end, a specific embodiment of the present disclosure involves the development of a highly predictive polygenic risk score (PGS) for GHDs using genetic data from the UK Biobank (UKBB) (Ref. 14).

As database repositories with genomics and non-genomics data on pregnancies are growing, PGS and composite risk scores for GHDs will be updated by comparing cases (pregnancies with GHDs) with controls using machine learning methodologies.

The PGS can further be integrated into clinical practice to provide a more accurate early assessment of risk for these disorders.

The role of dietary factors in the prevention of GHD has been suggested by several studies. For example, higher total energy and lower magnesium and calcium intake during pregnancy are associated with GHD (Ref. 9). Similarly, the consumption of additional salt in the diet has also been found to be linked to GHD (Ref. 10).

Nutrient status is also directly associated with an increased risk of pre-eclampsia. For example, increased serum triglyceride and fatty acids, and reduced levels of serum calcium, vitamin C, magnesium, and zinc have been linked to an increased risk of pre-eclampsia (Ref. 11).

Vitamin D insufficiency or deficiency during pregnancy has also been identified as a risk factor for pre-eclampsia (Ref. 12). Additionally, some studies suggest that the VDR Bsml polymorphism is closely associated with a higher predisposition to hypertension, indicating the importance of genetic factors in the etiology of GHD (Ref. 13).

Overall, these findings suggest that monitoring and maintaining proper nutrient intake, including adequate levels of magnesium, calcium, and vitamin D, and limiting salt intake during pregnancy, may play a role in reducing the risk of GHD and pre-eclampsia. Furthermore, it is crucial to comprehend genetic predispositions to identify women who might have an elevated risk of developing GHDs.

Among modifiable risk factors, physical activity and dietary intake before and during early pregnancy are of chief importance. The implementation of lifestyle modifications reduces the risk of gestational hypertension and pre-eclampsia.

Thus, identifying women at higher risk of GHD based on genetics and other factors and providing them with actionable nutritional and lifestyle recommendations to minimize risks is crucial. Identification should occur either at the preconception stage or during the first trimester of pregnancy.

### PRIOR ART

Few disclosures to date address assessing risks of gestational hypertensive disorders (GHD), and preeclampsia in women. A 2012 Chinese disclosure CN102373285A entitled “Noninvasive gene assay kit for female gestational hypertension” provides risk assessment for gestational hypertension based on four single nucleotide polymorphisms (SNPs). This SNP list is far from exhaustive, and the risk is not quantifiable.

Other prior disclosures are based on measuring the risk of pre-eclampsia and other GHDs using either proteins and peptides or metabolites. For example, a previous Japanese disclosure JP6185532B2 entitled “Detection of the risk of preeclampsia” provided a panel of metabolites related to the early (11-17 weeks) prediction of the risk of pregnant women developing preeclampsia before developing normal clinical symptoms.

Similarly, another previous disclosure, U.S. Pat. No. 8,932,823 B2, entitled “Methods for determining the risk of prenatal complications” determines the risk of preeclampsia in a pregnant woman. This US patent teaches a measuring of proteins (placental growth factor and pregnancy-associated plasma protein A).

Australian publication AU2020201695B2 entitled “Biomarkers and methods for predicting preeclampsia” may be relevant. This publication teaches the use of a panel of protein and peptide biomarkers for determining the probability of preeclampsia in pregnant females.

While earlier disclosures may provide metabolic or protein panels for assessment of a woman's risk of preeclampsia, they do not provide tools for assessing this risk before pregnancy, or in the first trimester. These disclosures also do not provide a platform for actionable nutritional and lifestyle recommendations to women who have a higher risk of GHDs. There are hence shortcomings regarding the assessment of GHD risks.

### BRIEF DESCRIPTION OF THE FIGURES

FIG. 1 is a block diagram of a system according to an embodiment of the present disclosure.

FIG. 1a is a block diagram of a system according to an embodiment of the present disclosure.

FIG. 2 is a block diagram of a system according to an embodiment of the present disclosure.

FIG. 2a is a block diagram of a system according to an embodiment of the present disclosure.

FIG. 3 is a table listing ID numbers for genetic variants (SNPs), weight coefficients, and nearby genes for gestational hypertension PGS.

FIG. 4 is a table listing ID numbers for genetic variants (SNPs), weight coefficients, and nearby genes for preeclampsia PGS.

### DETAILED DESCRIPTION

Systems and methods provided herein identify women at risk of gestational hypertensive disorders (GHD) based on genomics and other factors including biomarkers and demographics. The shortcomings of previous approaches described at least above are addressed herein by a dynamic self-learning system that identifies women at high risk for GHD and provides them with personalized dietary and lifestyle recommendations to reduce the risk of GHD.

Systems and methods provide personalized nutrition advice in the stages of preconception and early pregnancy. The advice is tailored to an individual woman's genomics data and other considerations that may be critical to ensure the health and wellness of mothers and babies.

The present disclosure provides for collecting large amounts of heterogeneous data from pregnant women that can serve as a basis for longitudinal studies. A principal objective herein is to improve the assessment of risk predisposition to GHDs and to further provide personalized nutrition and lifestyle recommendations that minimize the incidence of GHDs.

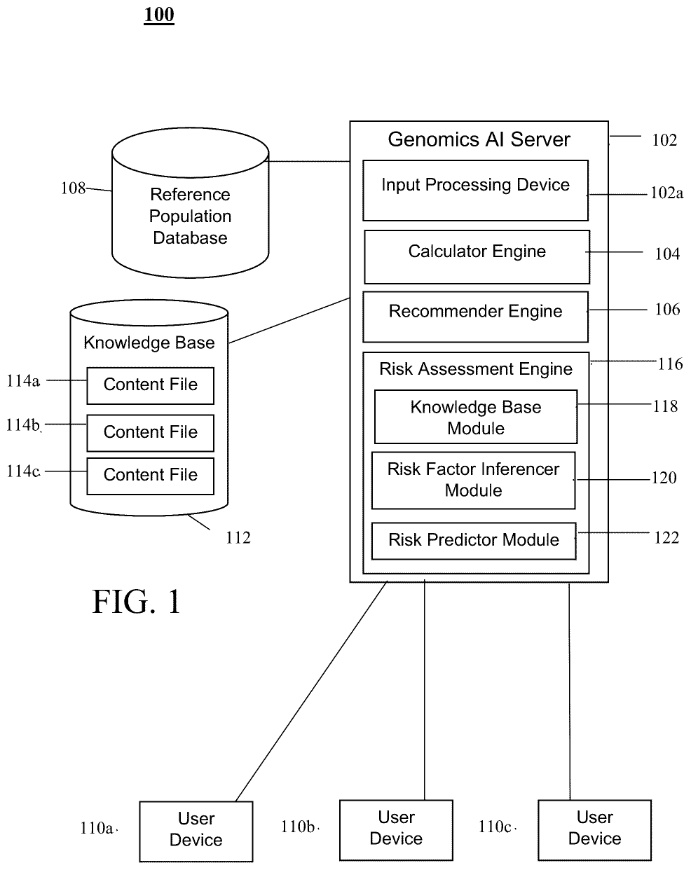

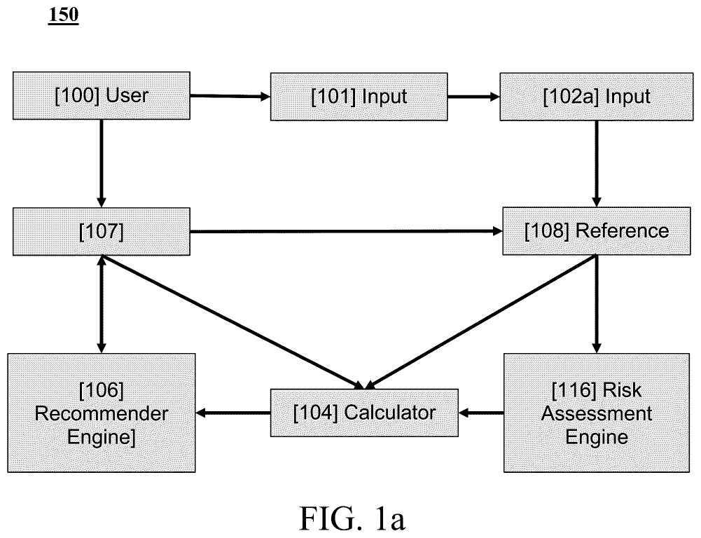

Turning to the figures, FIG. 1 illustrates the components and interactions of a system 100 for assessing risk predisposition to GHDs and developing personalized nutrition plans for use during stages of preconception, pregnancy, and lactation/postpartum according to an embodiment of the present disclosure.

System 100 comprises a genomics AI server 102 (that includes a calculator engine 104, a recommender engine 106, and risk assessment engine 116), knowledge base 112, and a reference population database 108. The system 100 also comprises a plurality of user devices 110a-c used by persons to submit data to the genomics A1 server 102 and to receive dietary programs and other data from the genomics AI server 102 and other components.

While quantity three user devices 110a-c are depicted in FIG. 1 and provided by the system 100, in embodiments more than or less than quantity three user devices 110a-c may be provided. System 100 also comprises a knowledge base 112 which contains content files 114a-c.

System 100 also comprises a risk assessment engine 116. The risk assessment engine 116 comprises a knowledge base module 118, a risk factor inferencer module 120, and a risk predictor module 122.

The Genomics AI server 102 may be a single computer or multiple physical computers situated at one or multiple geographic locations. While the calculator engine 104, the recommender engine 106, and the risk assessment engine 116 are depicted in FIG. 1 as contained by or components of the Genomics AI server 102 and executing on the Genomics AI server 102, in embodiments the calculator engine 104, the recommender engine 106, and the risk assessment engine 116 may be separate components or software executing on separate devices proximate or remote from the Genomics AI server 102.

While referred to as engines, the calculator engine 104, the recommender engine 106, and the risk assessment engine 116 may be combinations of hardware and software applications or entirely software applications. Components described herein as modules, submodules, or devices may be physical devices, combinations of a physical device and software, or entirely software. For example, the knowledge base module 118, the risk factor inferencer module 120, and the risk predictor module 122 may be combinations of hardware and software or primarily software.

The Genomics AI server 102 receives genomics and non-genomics data from women using the user devices 110a-c. The received data is processed by an input processing device 102a of the Genomics AI server 102 and stored in the reference population database 108. The received data is also provided to the calculator engine 104 to determine macro-and micro-nutrient needs and ranges for the woman.

Based on the macro-and micro-nutrient needs and ranges determined by the calculator engine 104, the recommender engine 106 generates dietary recommendations, foods, and recipes. Feedback regarding liking/disliking dietary recommendations and adverse effects such as morning sickness, or nausea, may be provided back to the recommender engine 106, the calculator engine 104, the reference population database 108, and the risk assessment engine 116 to improve future personalized dietary recommendations. The calculator engine 104 and the recommender engine 106 may rely on at least one algorithm to complete their tasks.

Additional data, such as the rate of weight gain during pregnancy, weight loss postpartum, blood pressure, glucose levels, and measurements of other biomarkers, may be collected from the woman and submitted to the components of the system 100 to improve future personalized dietary recommendations, and to discover relationships between genetics, genomics, dietary consumption, and preconception, pregnancy, lactation/postpartum traits.

FIG. 1a is a flow drawing depicting components of a system 150 according to an embodiment of the present disclosure. The components of the system 150 and depicted in FIG. 1a correspond to the components provided by the system 100 and depicted in FIG. 1. FIG. 1a illustrates the system 150 of receiving genomic and non-genomic data from a user, computing, via a machine learning methodology, risk predisposition to GHDs for an individual woman, further calculating personalized calories, including macro-and micro-nutrient needs and ranges based on the received data, and generating personalized dietary and lifestyle recommendations.

As shown in FIG. 1a, an input device [101] of system 150 is used to receive genomics and non-genomics data for females from a user module [100]. The received data is then processed by an input processing device [102a] and added for storage into reference storage [108]. The received data is thereafter provided to a risk assessment engine [116] to compute risk predisposition to GHDs for the individual female, and further calculate, via a calculator engine [104], personalized calories, macro-and micro-nutrient needs and ranges for the individual woman.

Based on the calculated calories, macro-and micro-nutrient needs, and ranges, a recommendation engine [106] generates dietary recommendations, foods, and recipes. Feedback regarding liking/disliking recommended foods and recipes, and adverse effects such as morning sickness and nausea may be provided [107] back to the recommendation engine [106], the calculator engine [104], and the reference storage [108] to improve future recommendations.

Data on pregnancy complications, including GHDs, pre-eclampsia, eclampsia, miscarriage, labor, birth details, and baby weight may be collected from the individual woman. Additional data, such as blood pressure, glucose levels, measurements of other biomarkers, rate of weight gain during pregnancy, or weight loss postpartum, may be collected from the individual woman and transmitted to the risk assessment engine [116], the calculator engine [104], and the reference storage [108].

These actions are intended to improve, via a machine learning methodology, the assessment of risk predisposition to GHDs, as well as other pregnancy-related traits and complications, and to further improve future personalized dietary recommendations. Further, this longitudinal data is utilized to discover associations and to build, via a machine learning methodology, predictive comprehensive models for pregnancy-related traits and pregnancy complications, taking into consideration genomics, and other data, including dietary consumption and lifestyle during preconception, pregnancy, and postpartum stages.

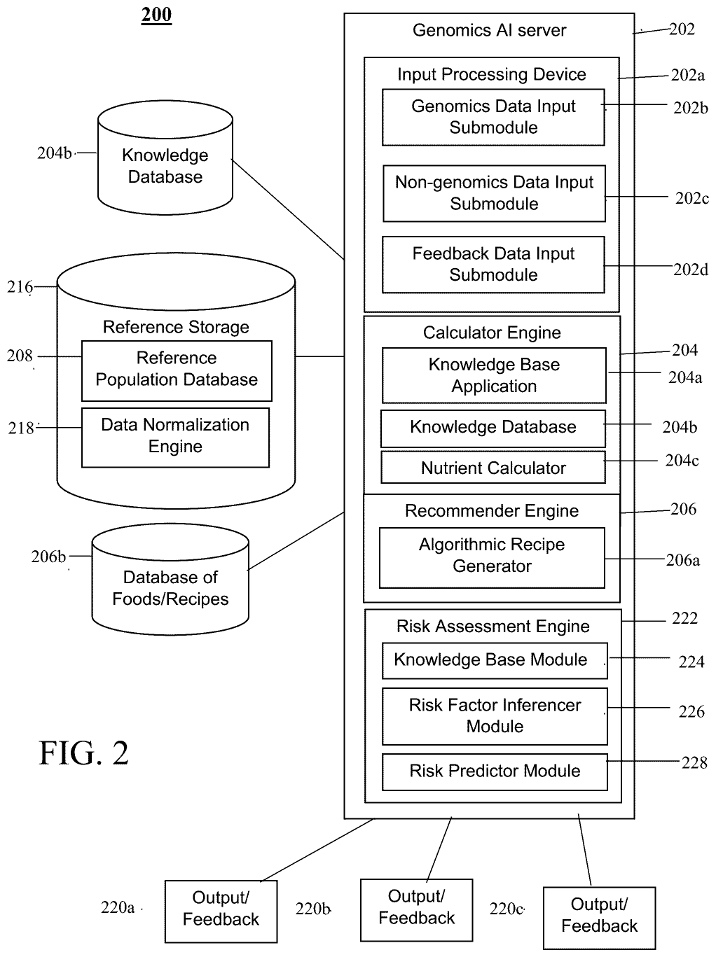

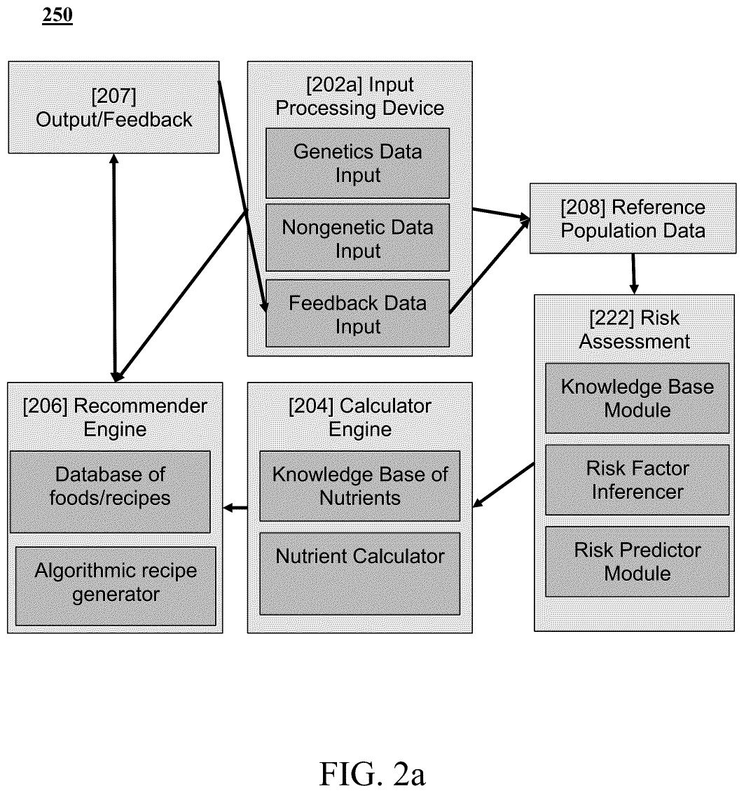

FIG. 2 is a block diagram depicting a system 200 provided by an embodiment of the present disclosure. Components of system 200 are partially indexed to components of system 100.

A Genomics AI server 202 is provided. An input processing device 220a, reference population database 208, calculator engine 204, and recommender engine 206 are provided by the system 200 as in the system 100.

System 200 also comprises a risk assessment engine 222. The risk assessment engine 222 comprises a knowledge base module 224, a risk factor inferencer module 226, and a risk predictor module 228.

Input processing device 202a includes three submodules or applications comprising a genomics data input submodule 202b, a non-genomics data input submodule 202c, and a feedback data input submodule 202d. In various embodiments, data input is done via an Internet web connection, via mobile application at home, and/or in an outpatient clinical environment.

Genomics AI server 202 receives genomics data from various sources via genomics data input submodule 202b that may be integrated with external information providers. In some embodiments, input data may be a file with genotype data uploaded by an individual, by an external genotyping or sequencing service/company using a generic or proprietary application programming interface (API), or by a third party, for example, physicians, healthcare providers, or dieticians. In other embodiments, input data is a file with RNA expression data or protein abundance data. This data may be uploaded by an individual, by an external sequencing service/company using generic or proprietary API, or by a third party, for example, physicians, or dieticians. Genomics data is pre-processed and may be analyzed using bioinformatics methods.

Genomics AI server 202 receives non-genomics data from various sources via the non-genomic data input submodule 202c. Non-genomics data may include data describing a woman's age, ethnicity, preconception/pregnancy/postpartum stage, demographics, height, weight, activity level, diet, habits, lifestyle, medical history, geolocation, environment, and preferences. Non-genomics data may contain data from physiological tests, for example, blood, urine, and stool, data from wearables, sensors, imaging data from professional devices or smartphones, and other relevant devices.

The non-genomics data input submodule 202c, which may be partially integrated with external information providers, enables input of non-genomics information by generic or proprietary API from imaging devices, sensors, wearables, and other relevant devices, or third-party expert reports, for example, physicians, dieticians, and aestheticians. The non-genomics data input submodule 202c submodule also enables self-reported questionnaires or data input by third parties.

The feedback data input submodule 202d is utilized when a woman provides reviews, survey responses, or other feedback to the system 200 about a dietary program provided to the woman. The feedback data input submodule 202d receives feedback from the woman about specific recipes and food recommendations and likes/dislikes. The feedback data input submodule 202d may also receive reports of adverse effects such as morning sickness, nausea, weight gain during pregnancy or weight loss postpartum, blood pressure, pregnancy complications, baby gestational age, baby weight, and lactation issues.

Upon receipt of genomic and non-genomic data, input processing device 202a shares the received data with a reference population database 208 which is a repository of genomic and non-genomic data for many expectant women and postpartum women as well as women in preconception.

The reference population database 208 may be a component of reference storage 216. Data stored in the reference population database 208 is continuously updated with new entries received from women at various stages of preconception, pregnancy, and postpartum. The reference population database 208 can also be updated by bulk downloads of genomic data from multiple women, by non-genomic data from third parties, and with material from databases and other repositories not associated with the system 200.

Feedback data, received from women at various stages of preconception, pregnancy, or postpartum, or from other parties, is also propagated to the reference population database 208, and, after processing with data normalization engine 218, may also be further transmitted to the calculator engine 204 and recommender engine application 206 to improve assessment of needs and future personalized dietary recommendations, foods, and recipes.

A continuous self-learning system may thereby be set into place. For example, by analyzing via computational algorithms and collected data, the system may infer that women with specific genetic variations are more likely to have more morning sickness in the first trimester if they consume specific foods. Similarly, the system may learn that specific foods and recipes help women deal with morning sickness and nausea.

The reference storage 216 is an information source for computing personalized macro-and micro-nutrient needs and ranges performed by the calculator engine 204. As with the reference population database 108 of the system 100, the reference population database 208 stored in reference storage 216 and provided by the system 200 may be a single database or multiple databases situated at a single or multiple geographic locations.

The calculator engine 204 comprises a knowledge base application 204a that organizes and dynamically structures state-of-the-art information and data in a knowledge database 204b related to macro-and micro-nutrients effects on the various stages of preconception, pregnancy, and postpartum/lactation. The knowledge database 204b contains data on at least:
(i) levels of macro-and micro-nutrients at different stages of preconception, pregnancy, and lactation/postpartum as recommended, by ob-gyns and nutritionists;
(ii) effects of medical history, age, biometric data, lifestyle factors, and dietary restrictions on levels of macro-and micro-nutrients at different stages of preconception, pregnancy, and lactation/postpartum; and
(iii) effects of genetic variations on levels of macro-and micro-nutrients at different stages of preconception, pregnancy, and lactation/postpartum.

Calculator engine 204 receives genomics and non-genomics data from the input processing device 202a. Calculator engine 204 compares the individual genomics and non-genomics data to stored data in the reference population database 208.

Calculator engine 204 computes personalized macro-and micro-nutrient risk likelihood predispositions, needs, and ranges based on the received data and material stored in knowledge base 204b. The calculator engine 204 may perform the computations of risk likelihood predispositions, needs, and ranges using at least one algorithm that may be proprietary and/or developed by a third-party source. The calculator engine 204 also includes a nutrient calculator 204c that calculates macro-and micro-nutrients for various dietary recommendations and meal plans and furnishes this information to other components of the system 200 as necessary.

The computed macro-and micro-nutrient needs and ranges for the individual woman are transmitted to the recommender engine 206 to generate dietary recommendations, foods, and recipes. The input processing device 202a may transmit additional data collected from the woman to the recommender engine 206.

In some embodiments, additional data collected from the woman may comprise dietary restrictions, preferences, allergies, and sensitivities. The recommender engine 206 algorithmically generates dietary recommendations, foods, recipes, daily and weekly meal planners, food shopping lists, and dietary tips. These actions may be based on the calculated macro-and micro-nutrient needs and additional data transmitted from the input processing device 202a.

The recommender engine 206 has access to a database of foods and recipes 206b. By performing multi-variable optimization, the recommender engine 206 generates weekly shopping lists and meal plans for a woman based on the woman's ranges for macro-and micro-nutrients, dietary restrictions, food allergies and sensitivities, and personal preferences. The recommender engine 206 relies upon an algorithmic recipe generator 206a to assist in creating recipes.

The database of foods and recipes 206b may have contents contributed, via feedback, by women who have been using the system 200. In embodiments, the recipes and foods are curated, via feedback, by women who have been using the system 200. For example, a specific recipe may be upvoted for women who have morning sickness during the first trimester of pregnancy. The recommender engine 206 and other components have access to reference storage 216 which hosts the reference population data 208 and an ML discovery module as a self-learning system (not shown in FIG. 2).

Feedback submitted by women at various stages of preconception, pregnancy, or postpartum, may be collected and submitted via a submodule that collects responses from provided dietary recommendations. Specifically, the feedback data may comprise liking/disliking dietary recommendations, foods, and recipes. Feedback data related to dietary recommendations, foods, and recipes are stored in a database of foods/recipes 206b.

The feedback data may then be transmitted to the reference population data 208, and, after processing, be further transmitted to the calculator engine 204 and to the recommender engine 206 to improve future personalized dietary recommendations, foods, and recipes. As noted, a continuous self-learning system may thereby be set in place.

The system 200 also comprises components depicted and enumerated as output/feedback 220a-c which may be equivalent or similar to the user devices 110a-c provided by the system 100 and depicted in FIG. 1. Output/feedback 220a-c receive dietary programs and provide feedback to the Genomics AI server 202. While quantity three output/feedback 220a-c are depicted in FIG. 2 and provided by the system 200, in embodiments more than or less than quantity three output/feedback 220a-c may be provided.

FIG. 2a is a block diagram depicting components of a system 250 according to an embodiment of the present disclosure. System 250 comprises an input processing device [202a], a reference population data [208], a risk assessment engine [222], a calculator engine [204], and a recommender engine [206]. These components may correspond to similarly named components of the system 200 depicted in FIG. 2.

The input processing device [102] receives genomics data and non-genomics data from female users. The input processing device [102] consists of three components: a genomics data component, a non-genomics data input component, and a feedback data input component. In some embodiments, data input by users is done via an Internet web connection, via a mobile application at home, and/or via an outpatient clinical environment.

The input processing device [102] receives genomics data (genetics data) from various sources via the genomics data component that is integrated with external information providers. In some embodiments, input data may be a file with genotype data uploaded by an individual, by an external genotyping function, by a sequencing service/company using a generic or proprietary application programming interface (API), or by third parties, for example, physicians, or dieticians. In some embodiments, input data is a file with RNA expression data or protein abundance data. Genomics data (DNA, RNA, or proteomics) is pre-processed and analyzed using bioinformatics methods directed to obtaining quantifiable results to enable further assessments.

The input processing device [102] receives non-genomic data from various sources via the non-genomics data input submodule. Non-genomic data may include data about an individual's age, height, weight, demographics, diet (including allergies, sensitivities, restrictions, and preferences), preconception/pregnancy/postpartum stage, physical activity level, medical history, geolocation, environment, and general concerns. Non-genomic data may contain data from clinical laboratory tests of serum, urine, and stool. Non-genomics data may further contain data from wearables, sensors, and imaging data from professional devices or smartphones, and other relevant devices.

The non-genomics data input submodule, which may be integrated with external information providers, enables input of non-genomics information by generic or proprietary API from relevant devices, including sensors, wearables, imaging devices, or third-party expert reports, for example. physicians, healthcare providers, and dieticians. The non-genomics data input submodule also enables self-reported questionnaires or data input by third parties.

The feedback data input submodule 207 is utilized when the user receives output from the system regarding optimal levels of macro-and micronutrients, food, and recipe recommendations. In some embodiments, the feedback data input submodule is used by the user to report data related to specific recipes, food recommendations, and likes/dislikes. The feedback data input submodule 207 may also be used by the user, or the third party, for example, a physician, to report adverse effects such as morning sickness, nausea, food cravings, weight gain during pregnancy, or weight loss postpartum, blood pressure, pregnancy complications, baby gestational age, baby weight, and lactation issues.

Upon receipt of genomic and non-genomic data, the input processing device [202a] propagates the received data to reference storage [208] which is a repository of genomic and non-genomic data for a plurality of individual females. The data stored in reference storage [208] is continuously updated with new entries received from individuals via the input processing device [202a]. Reference storage [208] can also be updated by bulk downloads of genomic data from multiple individual females as well as non-genomic data from external sources, data repositories, and third parties.

Feedback data, received from the user [207], is propagated to the reference storage [208]. After processing, using various data analysis tools, the feedback data is provided to the risk assessment engine [222], the calculator engine [204], and the recommender engine [206], to improve predictive models for assessment of the risk predisposition to GHDs.

Based on processing by at least the risk assessment engine [222], the feedback data may be used to further optimize and fine-tune dietary and lifestyle recommendations. A continuous self-learning system may thereby be set into place. By analyzing collected data, via machine learning methodologies, the system may improve the predictive model for assessment of the risk predisposition to GHDs.

The system may further build predictive models and assess risk predispositions to other pregnancy-related complications and side effects. The system may further infer that women with specific combinations of genetic variations, for example, those that are associated with higher levels of clinically measured biomarkers, such as glucose or albumin, or low levels of omega-3 fatty acids, are more likely to develop GHDs if they consume specific foods. Similarly, the system may learn that specific foods, for example, oily fish, or lifestyle changes may assist at-high-risk women to lower their chances of GHDs.

The reference storage [208] may provide bases for computing, via a machine learning methodology, a risk predisposition to GHDs for an individual female

performed by the risk assessment engine [222]. Reference storage [208] may further provide bases for computing personalized macro-and micro-nutrient predisposition risk likelihoods and needs performed by the calculator engine [204].

The risk assessment engine [222] consists of three modules: a knowledge base module, a risk factor inferencer module, and a risk predictor module. The knowledge base module integrates genomic and non-genomic data from the reference storage [208] with external knowledge on GHDs from published studies, randomized trials, and data repositories. The knowledge base module transforms this heterogeneous data from different sources into structured data using suitable computational methodologies and natural language processing tools.

The risk factor inferencer module infers, by applying various machine learning methodologies, from at least the knowledge base module, associations between various genomic and non-genomic factors and the risk of GHDs. Non-genomic risk factors include dietary intake, levels of clinically measured biomarkers and metabolites, physical activity, sleep, environmental factors, for example, pollution, and other phenotypic traits, for example, body mass index (BMI). The risk factor inferencer module computes, via at least a method of Mendelian randomization, genetic-based risk factors for GHDs utilizing

the data from at least the reference storage [208]. The risk factor inferencer module further validates inferred and genetically constructed risk factors of GHDs on the data from the reference storage [208].

The risk predictor module computes risk predisposition likelihoods to GHDs for women by integrating genomic and non-genomic data with measured and inferred risk factors in a machine learning model. The risk predictor module may perform the computations of risk predisposition using at least one algorithm that may be proprietary and/or developed by a third-party source, for example, polygenic risk scores.

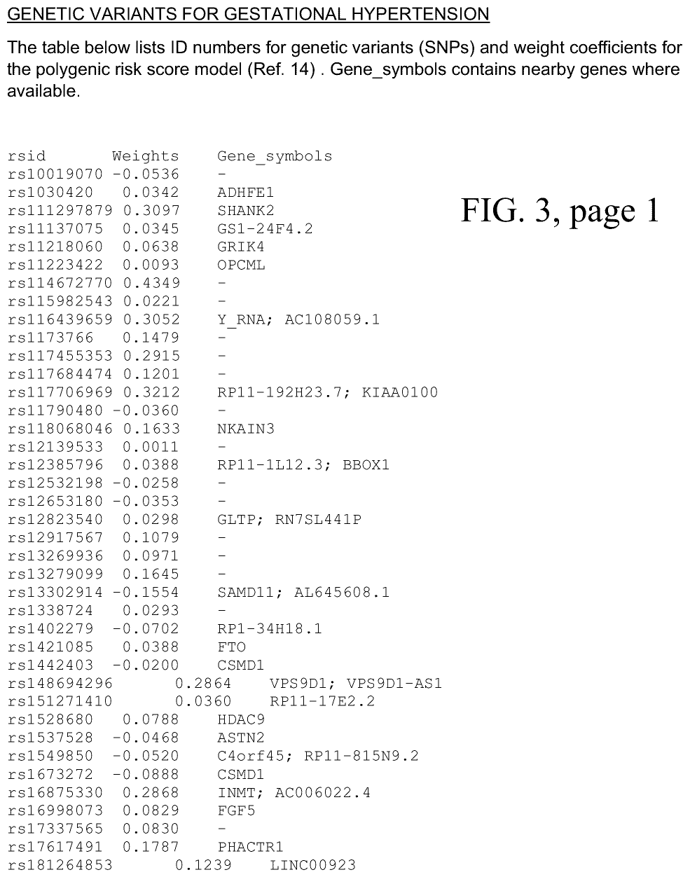

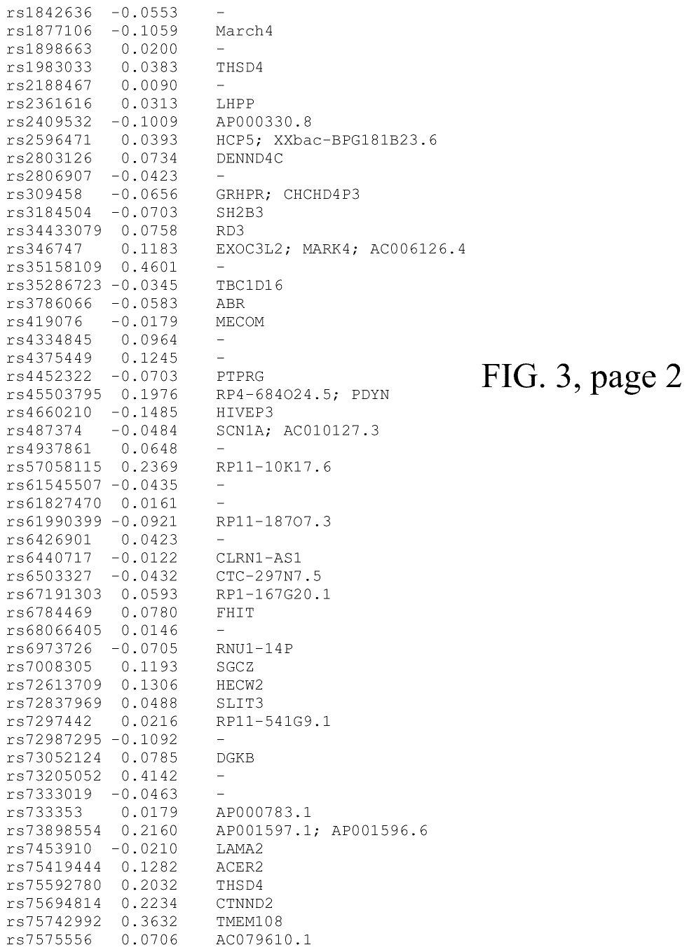

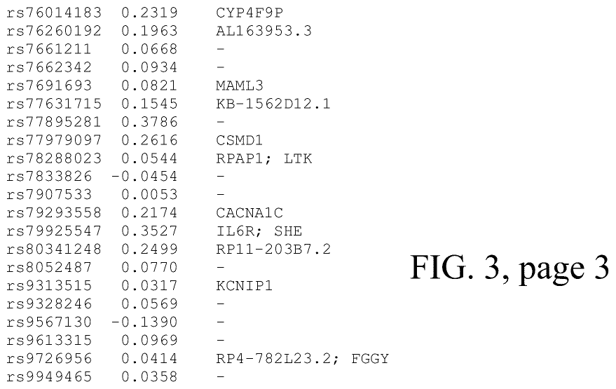

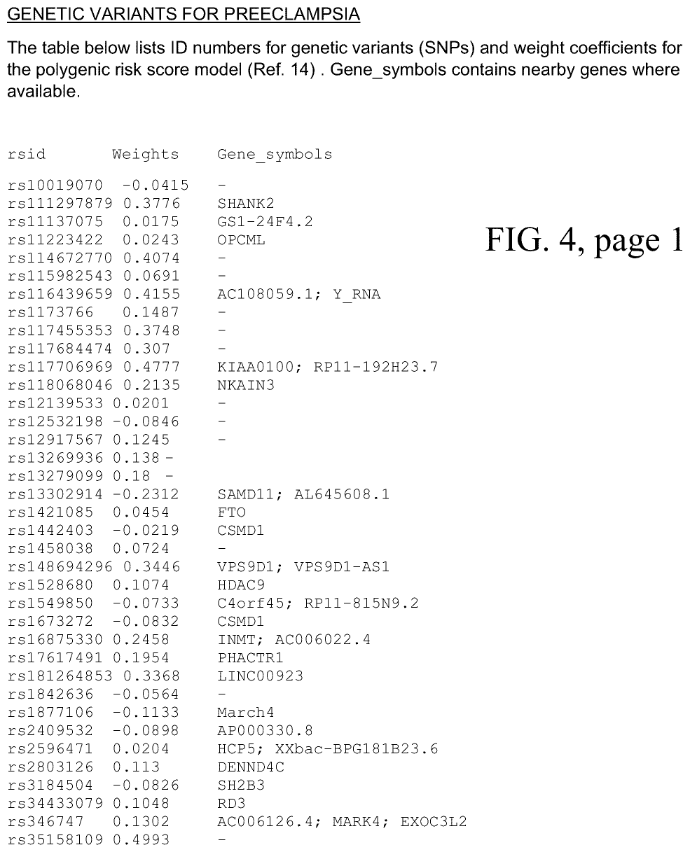

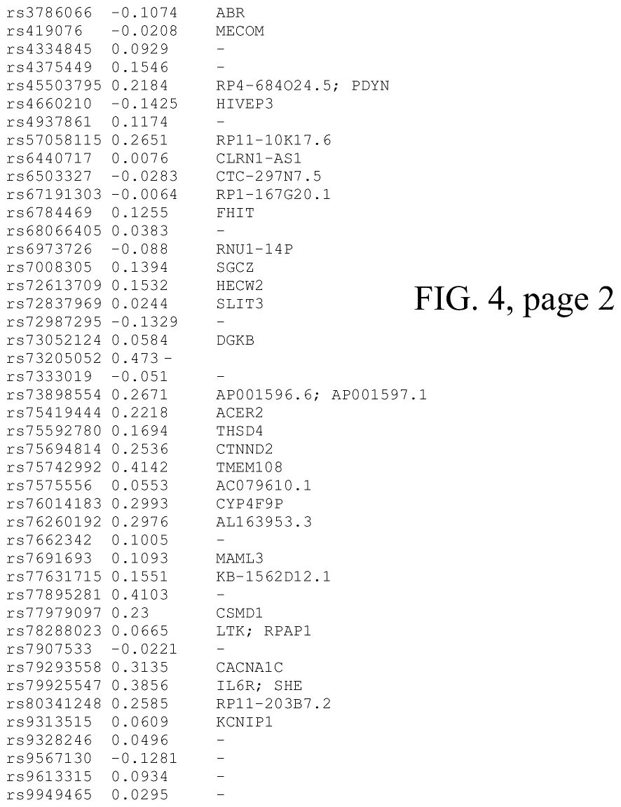

In an embodiment, a polygenic risk score (PGS) for gestational hypertension and a PGS for preeclampsia were developed using machine learning (REF. 14). SNPs and their corresponding weights for gestational hypertension are provided in a Table in FIG. 3. SNPs and their corresponding weights for preeclampsia are provided in a Table in FIG. 4. The tables in FIG. 3 and FIG. 4 contain columns with rs identifiers, weight coefficients, and nearby genes.

In an embodiment, the risk predictor module applies a supervised machine learning model to integrate genomic and non-genomic data from the reference storage

. The risk predictor module uses best practices of machine learning to develop and validate risk predisposition predictions utilizing the cases and controls for GHDs. In some embodiments, the machine learning model can be a generalized linear model, a classification model, a Bayesian model, a Neural Network Analysis (NNA), or an ensemble of several models.

In an embodiment, risk predisposition likelihood to GHDs is computed based on genetics using a polygenic risk score (PGS) model. Polygenic risk score (PGS) is a risk-weighted sum of the genetic variants, where the number of effect alleles is represented by either 0, 1, or 2. It is computed using a machine learning procedure (Ref. 14). As noted above, the polygenic risk score for GHDs is represented by SNPs, and their weight coefficients as provided in a Table in FIG. 3 (Ref. 14). Polygenic risk score for preeclampsia (considered separately) is represented by SNPs, and their weight coefficients as provided in a Table in FIG. 4. As noted, the tables in FIG. 3 and FIG. 4 contain columns with rs identifiers, weight coefficients, and nearby genes.

The risk predictor module optimizes predictions for risk predisposition to GHDs. It further calculates, via a suitable computational methodology, a proportion of risk from genomics, diet, and lifestyle factors.

In one example, the risk factor inferencer module integrated publicly available summary statistics data on GHDs and genome-wide association studies on anthropometric measures, levels of biomarkers, and metabolites on the other hand By applying Mendelian Randomization (MR) computational methodology, and/or at least one machine learning methodology to this data, the risk factor inferencer module determined that genetically proxied anthropometric measures such as BMI, waist circumference, and body fat percentages, significantly and causatively increase the risk of GHDs (Ref. 14). Specifically, higher genetically proxied waist circumference increases the risk predisposition to GHDs by nearly a factor of two (Odds Ratio=1.87). Additionally, MR identified body fat percentage (OR=1.72), BMI (OR=1.69), and hip circumference (OR=1.44) as causal risk-factors for GHD and preeclampsia. Female-specific adiposity that reflects an increase in fat mass at the expense of lean mass is the contributing risk factor to preeclampsia and eclampsia (OR=1.46). Further, higher body size that describes a slightly disproportionate increase in body mass compared to height, resulting in higher BMI, increases the odds of preeclampsia by over 30% (OR=1.35), followed by waist-hip-ratio (OR=1.28). Weight-neutral abdominal fat deposition is also identified as a significant risk factor for GHDs (OR=1.26), and preeclampsia (OR=1.2).

MR analyses confirm that genetically proxied elevated blood glucose levels causally increase the risk of GHDs by more than 20%. On the other hand, high levels of omega-3 fatty acids and docosahexaenoic acid causally lower the odds of GHDs by 25% (OR=0.75). Adequate levels of other fatty acid metabolites, including a long-chain fatty acid 10-nonadecenoate, monounsaturated omega-9 fatty acid, oleic acid, and adrenate, a very long-chain fatty acid, also lower the risk of GHDs and preeclampsia. Higher genetically constructed high-density lipoprotein cholesterol causally lowers the incidences of GHD, while low-density lipoprotein cholesterol causally increases the risk by 15%.

Both low and high maternal hemoglobin was found to be associated with adverse maternal outcomes, including preeclampsia. The MR analysis provides evidence that genetically proxied iron anemia increases the risk of gestational hypertension (OR=1.58), and preeclampsia (OR=1.36). Furthermore, genetically proxied total iron-binding capacity (TIBC) that rises when iron levels are low, has an increasing effect on the odds of preeclampsia or eclampsia. Transferrin saturation which is the ratio of serum iron and TIBC, and therefore becomes very low at iron anemia, shows anti-correlation with the incidences of GHD with marginal statistical significance (OR=0.85). These results align with the findings on the effect of iron deficiency anemia on the risk of GHD.

Taken together, these results further underscore the importance of maintaining a healthy BMI/WHR, low adiposity, and abdominal fat deposition, maintaining normal glucose levels, and iron levels, and consuming healthy fats. These findings provide a roadmap for identifying risk factors of GHDs, developing practical tests to measure risk-elevated biomarkers, and most importantly early screening that identifies women at higher risk of GHDs at the preconception or early pregnancy stages before its onset allowing comprehensive monitoring and preventative programs to mitigate the risks.

The calculator engine [204] receives genomics and non-genomics data

from at least the reference population module [208]. The calculator engine [204] comprises two modules: a knowledge base of nutrients and a nutrient calculator. The knowledge base module organizes and dynamically structures state-of-the-art knowledge related to macro and micronutrients at each stage of preconception, pregnancy, and postpartum/lactation.

The knowledge base of nutrients contains data on:
(i) levels of micro-and macronutrients required at different stages of preconception, pregnancy, and lactation/postpartum as recommended by obstetrician-gynecologists and nutritionists;
(ii) effects of medical history, age, biometric data, lifestyle factors, and dietary restrictions on levels of micro and macronutrients required at different stages of preconception, pregnancy, and lactation/postpartum; and
(iii) effects of genetic variations on levels of micro-and macronutrients that are required at different stages of preconception, pregnancy, and lactation/postpartum.

The knowledge base further comprises data on:
(i) micro-and macronutrients that may decrease the risk of GHDs (e.g., vitamin D, mono-unsaturated fats);
(ii) micro-and macro-nutrients that may elevate the risk of GHDs (e.g., glucose, unhealthy fats);
(iii) biomarkers that are associated with an elevated risk of GHDs; and biomarkers that are associated with a lower risk of GHDs;
(iv) micro-and macro-nutrients and foods that affect levels of risk-elevating biomarkers for GHDs, for example, leptin;
(v) micro-and macro-nutrients that affect levels of risk-decreasing biomarkers for GHDs (e.g. adiponectin).

The nutrient calculator of the calculator engine computes personalized micro-and macro-nutrient predisposition risk likelihoods, needs, and ranges based on the received data of an individual, and the knowledge base, by comparing the individual genomics and non-genomics data to the reference population data.

A predisposition risk likelihood of a specific macro-or micro-nutrient reflects levels of a said nutrient relative to the reference population, indicating either a risk of deficiency of the specific nutrient, for example, iron deficiency, or an overload of the said

nutrient, for example, iron. The calculator engine [204] may perform computations of predisposition risks, needs, and ranges using at least one algorithm that may be proprietary and/or developed by a third-party source, for example, a polygenic risk score.

The system transmits computed macro-and micro-nutrient needs and ranges for the individual woman to the recommender engine [206] to generate dietary recommendations, foods, and recipes. The input processing device [202a] may transmit

additional data collected from the user to the recommender engine [206]. In some embodiments, additional data collected from the user may comprise dietary restrictions, preferences, allergies, and sensitivities.

The recommender engine [206] algorithmically generates dietary recommendations, foods, recipes, daily and weekly meal planners, food shopping lists, and nutritional tips. These actions are based on the calculated personalized macro-and micro-nutrient needs and additional data transmitted from the input processing device [202a].

The recommender engine [206] in embodiments has access, via API, to a large database of foods and recipes. By performing multi-variable optimization, the recommender engine [206] generates daily/weekly shopping lists and meal plans for individual women based on personalized risk predispositions to gestational hypertensive disorders, ranges for macro-and micro-nutrients, dietary restrictions, food allergies and sensitivities, and personal preferences.

In other embodiments, the recommender engine [206] may have access to a proprietary database of recipes, contributed, via feedback, by women who have been using the system. In embodiments, the recipes and foods are curated, via feedback, by women who have been using the system. For example, a specific recipe may be upvoted for women who have morning sickness during the first trimester of pregnancy.

Feedback may be provided by output/feedback [207] that collects responses from an individual woman on the provided dietary recommendations, shopping lists, and meal plans. Specifically, the feedback data may comprise liking/disliking dietary recommendations, foods, and recipes. Feedback data related to dietary recommendations, foods, and recipes are stored in the knowledge base of the recommender engine [206]. The feedback data may then be transmitted to reference storage [208], and, after processing, be further transmitted to the calculator engine [204] and to the recommender engine [206] to improve future personalized dietary recommendations, foods, and recipes. A continuous self-learning system may thereby be set in place.

In an embodiment, the recommender engine [206] may execute on a mobile computing device such as a smartphone or a tablet computing device. The recommender engine [206] hardware platform may be a desktop computing device or a laptop computing device. The recommender engine [206] hardware platform may include more than one computing device, such as output/feedback [207], configured to provide a user interface and one or more server computing devices configured to provide computational functionality such as the functionality of the calculator engine [204].

In such embodiments, the user computing device and one or more server computing devices may communicate via any suitable communication technology or technologies. Such technologies may comprise a wired technology comprising Ethernet, USB, or the Internet, or a wireless technology comprising WiFi, WiMAX, 3G, 4G, LTE, or Bluetooth.

In an embodiment, a system for computing risk predisposition to gestational hypertensive disorders for an individual woman is provided. The system comprises a computer and an application stored in the computer that when executed computes, based on received data, risk predisposition to gestational hypertensive disorders for a female. The system also calculates, based at least on the computed risk predisposition, calories, and macro-and micro-nutrient needs for the female. The system also generates, based on the calculations, dietary recommendations, foods, and recipes for the female.

The system further receives feedback from a plurality of females regarding liking/disliking of at least previous dietary recommendations comprising foods and recipes and receives further feedback regarding adverse reactions comprising at least one of morning sickness and nausea. The system further receives additional data comprising at least one of pregnancy complications, blood pressure, and glucose levels. The system further improves, based at least on the received feedback and the received additional data, and via at least a machine learning methodology, assessment of risk predisposition to gestational hypertensive disorders for at least the plurality of females.

The received data comprises genomics and non-genomic data describing women. The system performs the computations via at least one machine learning methodology.

Longitudinal data comprising at least the additional data is used to discover associations and to build, via at least one of the machine learning methodologies, predictive comprehensive models for at least one of pregnancy-related traits and pregnancy complications.

The discovery of associations and the building of the predictive comprehensive models is based at least on genomics data and non-genomics data comprising at least one of dietary consumption and lifestyle during preconception, pregnancy, and postpartum stages.

The system presents inquiries to the female in at least a questionnaire format, the inquiries comprising questions directed to at least one of liking/disliking recommended foods and recipes, adverse reactions to at least recommendations, and questions related to at least one of preconception, pregnancy, or postpartum/lactation.

The longitudinal data further comprises responses to inquiries regarding potential gestational hypertensive disorders development.

In another embodiment, a system for determining risk predisposition to gestational hypertensive disorders for a female is provided. The system comprises a computing device and an application executing on the computing device that receives feedback data reported by a plurality of users, the feedback related to specific food and dietary recommendations associated with at least mitigation of gestational hypertensive disorders. The system also receives user data comprising genomics data and non-genomics data from the plurality of users. The system also provides the feedback data and the user data to a risk assessment engine. The system also directs the risk assessment engine to adjust, based at least on the feedback data and the user data, predictive models for assessment of risk predisposition to gestational hypertensive disorders. The system also builds a continuous self-learning system that further refines assessments of risk predispositions based on ongoing adjustments of the predictive models and based on continuing receipt of feedback data and user data.

The feedback data further describes adverse reactions comprising at least one of morning sickness, nausea, food cravings, weight gain during pregnancy, weight loss post-partum, blood pressure, pregnancy complications, baby gestational age, baby weight, and lactation issues.

By analyzing collected data, via at least one machine learning methodology, the system supports the building of the continuous self-learning system.

The genomics data comprises at least one of DNA data, RNA expression data, protein abundances data, gut or oral microbiome data, measured by genotyping array, or next-generation sequencing. The non-genomics data comprises at least one of age, height, weight, ethnicity, medical history, diet, demographics, and stage, wherein stage comprises one of preconception, pregnancy, and postpartum.

In yet another embodiment, a method of determining genomics-based risk factors for gestational hypertensive disorders is provided. The method comprises a risk factor inferencer module of a risk assessment engine inferring associations between genomic and non-genomic factors and risk of gestational hypertensive disorders. The method also comprises the module, via at least a method of Mendelian randomization, and based at least on the inferred associations, computing genetic-based risk factors for gestational hypertensive disorders, using data from at least a reference storage. The method also comprises the module validating the inferred associations and validating genetic-based risk factors of gestational hypertensive disorders.

The method also comprises a risk predictor module of the engine computing risk predisposition likelihood to gestational hypertensive disorders for individual women by integrating genomic and non-genomic data and inferred and computed risk factors using a machine learning model.

The method also comprises the risk predictor module performing the computations using at least one algorithm, the algorithm at least one of proprietary and developed by a third-party source.

The method also comprises the risk predictor module applying a supervised machine learning model to integrated female data, the model comprising at least one of a generalized linear model, a classification model, a Bayesian model, and a Neural Network Analysis (NNA).

The method also comprises the risk predictor module calculating, via computational methodology, a proportion of risk from at least one of genomics, diet, and lifestyle factors.

The method also comprises a knowledge base module of the engine gathering heterogeneous information by integrating genomic and non-genomic data from the reference storage with external knowledge about gestational hypertensive disorders, the external knowledge comprising at least one of published studies, randomized trials, and data repositories.

The method also comprises the knowledge base module transforming the heterogeneous data into structured data using computational methodologies and natural language processing tools.

In embodiments, general steps of systems and methods provided herein may comprise:
1. The system receives an individual woman's genomics data and non-genomics data.
2. The system adds the woman's data to the population data and compares the woman's data to the population data.
3. The system collects longitudinal data on pregnancy progress and lactation that includes objective measurements and self-reported data from individual women. Objective data measurements include an individual woman's weight, age, medical data, physiological data, and wearables/sensors data measured at various time intervals. Objective data may contain reports from physicians and results from medical laboratories or at-home tests for pregnancy-related biomarkers. Self-reported data includes an individual woman's pregnancy complications, including gestational hypertensive disorders (GHD), gestational hypertension, or preeclampsia, symptoms (such as morning sickness, heartburn, nausea, and vomiting), food cravings, food intolerances, and sensitivities, liking or disliking specific foods and recipes, emotional wellbeing at various time-points.
4. The system integrates population genomics data with the population's non-genomics data.
5. The system propagates the individual woman's longitudinal data to the module with population data.
6. The system computes the risk likelihood (risk) predisposition to GHDs for the individual woman by utilizing machine learning methods and comparing an individual woman's data to the population data.
7. The system computes the individual woman's macro-and micro-nutrient needs and ranges based on pregnancy/lactation stage, calculated risk predisposition to gestational hypertensive disorders, activity level, age, genomics data, medical history, and physiological and other data by utilizing machine learning methods and comparing an individual's woman's data to the population data.
8. The system algorithmically generates personalized weekly shopping lists and meal plans by performing multi-variable optimization of food and recipe databases for the individual woman. These steps are based on her personalized ranges for macro-and micro-nutrients, dietary restrictions, food allergies and sensitivities, and personal preferences.
9. The system relies on a reporting and feedback module to send and receive data.

### REFERENCES

1. Umesawa and Kobashi. Epidemiology of hypertensive disorders in pregnancy: Prevalence, risk factors, predictors and prognosis. Hypertens Res. 2017; 40: 213-220
2. Lee et al. Pre-eclampsia: A Scoping Review of Risk Factors and Suggestions for Future Research Direction. Regen. Eng. Transl. Med. 2022, 8, 394-406
3. Wisner. Gestational hypertension and preeclampsia. MCN Am. J. Matern. Nurs. 2019, 44, 170
4. Stevens et al. Short-term costs of preeclampsia to the United States health care system. Am J Obstet Gynecol. 2017 September; 217(3): 237-248.e16. doi: 10.1016/j.ajog.2017.04.032. Epub 2017 Jul. 11. PMID: 28708975
5. Tan, M.; Wright, D.; Syngelaki, A.; Akolekar, R.; Cicero, S.; Janga, D.; Singh, M.; Greco, E.; Wright, A.; Maclagan, K.; et al. Comparison of diagnostic accuracy of early screening for pre-eclampsia by NICE guidelines and a method combining maternal factors and biomarkers: Results of SPREE. Ultrasound Obstet. Gynecol. 2018, 51, 743-750
6. MacDonald et al. Clinical tools and biomarkers to predict preeclampsia. EBioMedicine 2022, 75, 103780
7. Cnattingius et al. Maternal and fetal genetic factors account for most of familial aggregation of preeclampsia: A population-based Swedish cohort study. Am. J. Med Genet. Part A 2004, 130, 365-371
8. Buurma et al. Genetic variants in pre-eclampsia: A meta-analysis. Hum. Reprod. Update. 2013; 19: 289-303
9. Schoenaker et al. The association between dietary factors and gestational hypertension and pre-eclampsia: a systematic review and meta-analysis of observational studies. BMC Med. 2014 Sep. 22; 12: 157
10. Singh et al. Effects of diet on hypertensive disorders during pregnancy: A cross-sectional study from a teaching hospital. J Family Med Prim Care. 2021; 10(9): 3268-3272
11. Xu et al. Role of nutrition in the risk of preeclampsia. Nutr Rev. 2009; 67: 639-657; Patrelli et al. Calcium supplementation and prevention of preeclampsia: a meta-analysis. J Matern Fetal Neonatal Med. 2012; 25: 2570-2574; Conde-Agudelo et al. Supplementation with vitamins C and E during pregnancy for the prevention of preeclampsia and other adverse maternal and perinatal outcomes: a systematic review and meta-analysis. Am J Obstet Gynecol. 2011; 204: 503.e501-e512; Makrides and Crowther. Magnesium supplementation in pregnancy. Cochrane Database Syst Rev. 2001; 4: CD000937
12. Akbari et al. Association of vitamin D level and vitamin D deficiency with risk of preeclampsia: A systematic review and updated meta-analysis. Taiwan J Obstet Gynecol. 2018 April;57(2): 241-247; Cao et al. Vitamin D stimulates miR-26b-5p to inhibit placental COX-2 expression in preeclampsia. Sci Rep. 2021 11(1): 11168
13 Magiełda-Stola et al. The Significance of VDR Genetic Polymorphisms in the Etiology of Preeclampsia in Pregnant Polish Women. Diagnostics (Basel). 2021 Sep. 17; 11(9): 1698
14. Perišić et al. Polygenic Risk Score and Risk Factors for Gestational hypertensive disorders. J Pers Med. 2022. 12(9): 1381

What is claimed is:

1. A system for computing risk predisposition to gestational hypertensive disorders for an individual woman comprising:
a computer; and
an application stored in the computer that when executed:
computes, based on received data, risk predisposition to gestational hypertensive disorders for a female,
calculates, based at least on the computed risk predisposition, calories, and macro-and micro-nutrient needs for the female, and
generates, based on the calculations, dietary recommendations, foods, and recipes for the female.

2. The system of claim 1, wherein the system further:
receives feedback from a plurality of females regarding liking/disliking of at least previous dietary recommendations comprising foods and recipes and receives further feedback regarding adverse reactions comprising at least one of morning sickness and nausea,
receives additional data comprising at least one of pregnancy complications, blood pressure, and glucose levels,
improves, based at least on the received feedback and the received additional data, and via at least a machine learning methodology, assessment of risk predisposition to gestational hypertensive disorders for at least the plurality of females.

3. The system of claim 1, wherein the received data comprises genomics and non-genomic data describing women.

4. The system of claim 1, wherein the system performs the computations via at least one machine learning methodology.

5. The system of claim 1, wherein longitudinal data comprising at least the additional data is used to discover associations and to build, via at least one of the machine learning methodologies, predictive comprehensive models for at least one of pregnancy-related traits and pregnancy complications.

6. The system of claim 5, wherein the discovery of associations and the building of the predictive comprehensive models is based at least on genomics data and non-genomics data comprising at least one of dietary consumption and lifestyle during preconception, pregnancy, and postpartum stages.

7. The system of claim 1, wherein the system presents inquiries to the female in at least a questionnaire format, the inquiries comprising questions directed to at least one of liking/disliking recommended foods and recipes, adverse reactions to at least recommendations, and questions related to at least one of preconception, pregnancy, or postpartum/lactation.

8. The system of claim 5, wherein the longitudinal data further comprises responses to inquiries regarding potential gestational hypertensive disorders development.

9. A system for determining risk predisposition to gestational hypertensive disorders for a female, comprising:
a computing device; and
an application executing on the computing device that:
receives feedback data reported by a plurality of users, the feedback related to specific food and dietary recommendations associated with at least mitigation of gestational hypertensive disorders,
receives user data comprising genomics data and non-genomics data from the plurality of users,
provides the feedback data and the user data to a risk assessment engine,
directs the risk assessment engine to adjust, based at least on the feedback data and the user data, predictive models for assessment of risk predisposition to gestational hypertensive disorders, and
builds a continuous self-learning system that further refines assessments of risk predispositions based on ongoing adjustments of the predictive models and based on continuing receipt of feedback data and user data.

10. The system of claim 9, wherein the feedback data further describes adverse reactions comprising at least one of morning sickness, nausea, food cravings, weight gain during pregnancy, weight loss post-partum, blood pressure, pregnancy complications, baby gestational age, baby weight, and lactation issues.

11. The system of claim 9, wherein by analyzing collected data, via at least one machine learning methodology, the system supports the building of the continuous self-learning system.

12. The system of claim 9, wherein the genomics data comprises at least one of DNA data, RNA expression data, protein abundances data, gut or oral microbiome data, measured by genotyping array, or next-generation sequencing.

13. The system of claim 9, wherein the non-genomics data comprises at least one of age, height, weight, ethnicity, medical history, diet, demographics, and stage, wherein stage comprises one of preconception, pregnancy, and postpartum.

14. A method of determining genomics-based risk factors for gestational hypertensive disorders, comprising:
a risk factor inferencer module of a risk assessment engine inferring associations between genomic and non-genomic factors and risk of gestational hypertensive disorders;
the module, via at least a method of Mendelian randomization, and based at least on the inferred associations, computing genetic-based risk factors for gestational hypertensive disorders, using data from at least a reference storage; and
the module validating the inferred associations and validating genetic-based risk factors of gestational hypertensive disorders.

15. The method of claim 14, further comprising a risk predictor module of the engine computing risk predisposition likelihood to gestational hypertensive disorders for individual women by integrating genomic and non-genomic data and inferred and computed risk factors using a machine learning model.

16. The method of claim 15, further comprising the risk predictor module performing the computations using at least one algorithm, the algorithm at least one of proprietary and developed by a third-party source.

17. The method of claim 14, further comprising the risk predictor module applying a supervised machine learning model to integrated female data, the model comprising at least one of a generalized linear model, a classification model, a Bayesian model, and a Neural Network Analysis (NNA).

18. The method of claim 14, further comprising the risk predictor module calculating, via computational methodology, a proportion of risk from at least one of genomics, diet, and lifestyle factors.

19. The method of claim 14, further comprising a knowledge base module of the engine gathering heterogeneous information by integrating genomic and non-genomic data from the reference storage with external knowledge about gestational hypertensive disorders, the external knowledge comprising at least one of published studies, randomized trials, and data repositories.

20. The method of claim 19, further comprising the knowledge base module transforming the heterogeneous data into structured data using computational methodologies and natural language processing tools.
